(19) United States
(12) Plant Patent Application Publication
(10) Pub. No.: US 2017/0135265 P1
(43) Pub. Date: May 11, 2017

(54) IBERIS PLANT NAMED 'IBSZ0003'

(50) ;

(71) Applicant: SYNGENTA PARTICIPATIONS AG, Basel (CH)

(72) Inventor: Henricus Godefridus Wilhelm Stemkens, Andijk (NL)

(73) Assignee: SYNGENTA PARTICIPATIONS AG, Basel (CH)

(21) Appl. No.: 15/330,004

(22) Filed: Jul. 25, 2016

Related U.S. Application Data
(60) Provisional application No. 62/252,746, filed on Nov. 9, 2015.

(51) Int. Cl. A01H 5/02

(52) U.S. Cl. PLT/263.1

(57) ABSTRACT

A new Iberis plant named IBSZ0003′ particularly distinguished by its midsize pure white flowers that are early flowering, dark green foliage and a very compact, densely branched mounded plant habit.

Latin name of the genus and species of the plant claimed: Iberis sempervirens.

Varietal denomination: ‘IBSZ0003’.

### BACKGROUND OF THE NEW PLANT

The present invention comprises a new Iberis, botanically known as Iberis sempervirens, and hereinafter referred to by the variety name ‘IBSZ0003’.

‘IBSZ0003’ is a product of a planned breeding program. The new cultivar ‘IBSZ0003’ has midsize pure white flowers that are early flowering, dark green foliage and a very compact, densely branched mounded plant habit.

‘IBSZ0003’ originates from an open pollination in a greenhouse in April 2007 in Enkhuizen, The Netherlands. The female parent was an unpatented, proprietary plant designated ‘K1055-1’ with smaller white flowers, lighter green foliage and is less branched when compared to ‘IBSZ0003’.

The male parent of ‘IBSZ0003’ is unknown. The resultant seed was sown in August 2007 in Enkhuizen, The Netherlands.

‘IBSZ0003’ was selected as one flowering plant within the progeny of the stated cross in March 2008 in a greenhouse in Enkhuizen, The Netherlands.

The first act of asexual reproduction of ‘IBSZ0003’ was accomplished when vegetative cuttings were propagated from the initial selection in June 2008 in a greenhouse in Enkhuizen, The Netherlands.

### BRIEF SUMMARY OF INVENTION

Horticultural examination of plants grown from cuttings of the plant initiated in June 2008 in Enkhuizen, The Netherlands, and continuing thereafter, has demonstrated that the combination of characteristics as herein disclosed for ‘IBSZ0003’ are firmly fixed and are retained through successive generations of asexual reproduction.

‘IBSZ0003’ has not been observed under all possible environmental conditions. The phenotype may vary significantly with variations in environment such as temperature, light intensity and day length.

A Plant Breeder's Right for this cultivar was applied for in the European Union on May 28, 2015 and assigned No. 2015/1115. ‘IBSZ0003’ has not been made publicly available more than one year prior to the filing of this application.

The following traits have been repeatedly observed and are determined to be basic characteristics of the new variety. The combination of these characteristics distinguishes this Iberis as a new and distinct variety.

### BRIEF DESCRIPTION OF THE PHOTOGRAPHS

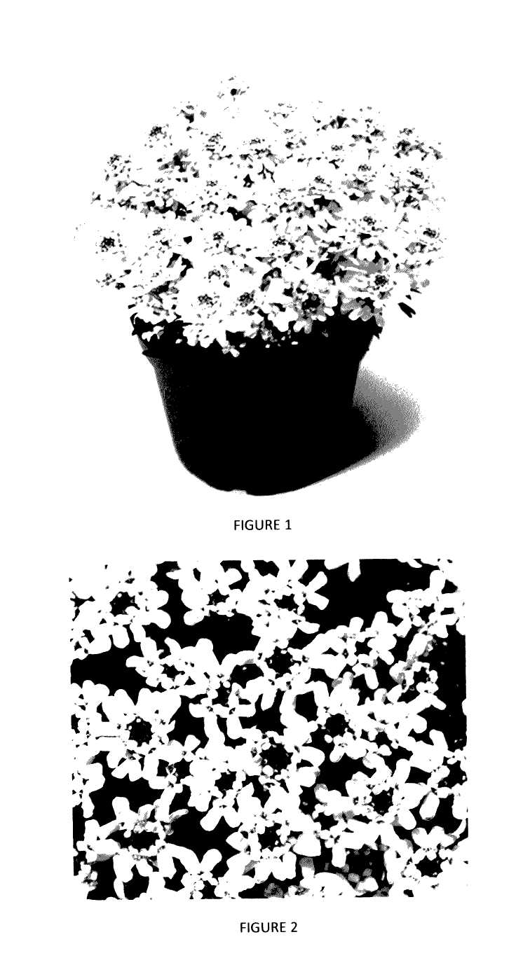

The accompanying photographic drawings show typical flower and foliage characteristics of ‘IBSZ0003’ with colors being as true as possible with an illustration of this type. The photographic drawings show in FIG. 1 a flowering plant of the new variety and in FIG. 2 a close-up of the inflorescences.

### DETAILED BOTANICAL DESCRIPTION

The plant descriptions and measurements were taken in Enkhuizen, The Netherlands under natural light in March 2015. These plants were approximately 34 weeks old and were growing in 13 cm pots in shade-house trials. The plants used for the photographs were about 34 weeks old growing in Enkhuizen, The Netherlands and the photographs were taken in March 2015.

Color Chart used: Royal Horticultural Society Colour Chart (R.H.S.) 2001

TABLE 1
DIFFERENCES BETWEEN THE NEW VARIETY ‘IBSZ0003’
AND A SIMILAR VARIETY

| | ‘IBSZ0003’ | ‘Snow Cone’ |
|---|---|---|
| Flower size: | Smaller | Larger |
| Plant size: | Smaller | Bigger |
| Branching habit: | More branching | Less branching and upright |

Plant:
   Form, growth and habit.—Dense, compact, mounded and branched.
   Plant height.—8.0-9.0 cm.
   Plant height (inflorescence included).—9.5-11.0 cm (without pot).
   Plant width.—18.0-22.0 cm.
Roots:
   Number of days to initiate roots.—About 9 days at 18 degrees C.
   Number of days to produce a rooted cutting.—3 weeks at 18 degrees C.
   Type.—Fine, fibrous, white, moderate branched.
   Color.—RHS N155B but whiter.
Foliage:
   Arrangement and attachment.—Alternate simple-decussate, sessile.
   Immature, leaf color, upper surface.—RHS 135 B.
   Lower surface.—RHS 141A.
   Mature, leaf color, upper surface.—RHS 135B.
   Lower surface.—RHS 141A.
   Length.—1.5 (juvenile)-3.5 (adult) cm.
   Width.—0.3 (juvenile)-0.5 (adult) cm.
   Shape.—Linear.
   Base shape.—Attenuate, clasped to stem.
   Apex shape.—Acute.
   Margin.—Entire.
   Texture, upper surface.—Punctuate.
   Lower surface.—Punctuate.
   Color of veins, upper surface.—RHS 144C/N144C.
   Color of veins, lower surface.—RHS N144B.
   Venation pattern.—Pinnate.
Stem:
   Quantity of main branches.—About 10-112, with each a few lateral branches.
   Color of stem.—RHS 141D.
   Length of stem.—4.5-7.0 cm (including peduncle).
   Diameter.—0.15 cm.
   Length of internodes.—0.5-0.7 cm.
   Texture.—Punctuate, no hairs.
Inflorescence:
   Type.—Short simple corymb.
   Quantity of inflorescences per plant.—More than 100.
   Inflorescence length.—About 4.0 cm.
   Inflorescence width.—2.5-3.0 cm at widest point.
   Lastingness of individual blooms on the plant.—About 14 days.
   Fragrance.—None.
Bud (just before opening/showing color):
   Color.—Closest to RHS 155A.
   Length.—0.2-0.3 cm.
   Width.—0.3 cm.
   Shape.—Globose.
   Pedicel color.—RHS 143B.
   Pedicel length.—0.7 cm.
   Pedicel diameter.—0.7 cm.
   Pedicel texture.—Smooth.
Corolla:
   Floret type.—4 petals—2 small and 2 large.
   Floret diameter.—1.0 cm.
   Floret depth.—0.65 cm.
   Petal (all) color, upper surface.—Whiter than RHS N155B.
   Lower surface.—Whiter than RHS N155B.
   Upper/shorter petals.—
   Length.—0.3 cm.
   Width.—0.3 cm.
   Shape.—Obovate to truncate.
   Apex shape.—Shallowly rounded.
   Margin.—Entire.
   Lower/longer petals.—
   Length.—0.7 cm.
   Width.—0.3 cm.
   Shape.—Obovate to truncate.
   Apex shape.—Shallowly rounded.
   Margin.—Entire.
   Petal (all) texture, upper surface.—Papillose.
   Lower surface.—Papillose.
Calyx:
   Quantity.—4 sepals.
   Color, upper surface.—RHS 143B.
   Lower surface.—RHS 143B.
   Length.—0.2 cm.
   Width.—0.1 cm.
   Shape.—Ovate-elliptic.
   Apex shape.—Acute.
   Base.—Fused.
   Margins.—Entire.
   Texture, upper/inner surface.—Lustrous.
   Lower/outer surface.—Lustrous.
Reproductive organs:
   Gynoecium.—
   Pistil quantity.—1.
   Length.—0.3-0.35 cm.
   Style color.—RHS 151C.
   Style length.—0.25-0.3 cm.
   Stigma color.—RHS 151A.
   Stigma shape.—Bi-lobed.
   Ovary.—Not very discernable.
   Androecium.—
   Stamens, quantity.—6; 4 longer, 2 shorter.
   Color of filaments.—RHS 154D.
   Length of longer filaments.—0.3 cm.
   Length of shorter filaments.—0.15 cm.
   Anther color.—RHS 154D.
   Anther length.—0.05 cm.
   Anther shape.—Oval.
   Color of pollen.—RHS 1B.
   Pollen amount.—Abundant.
   Fertility/seedset.—Neither seed nor fruit production has been observed.
   Disease/pest resistance.—Resistance to pathogens and pests common to Iberis has not been observed.
   Drought tolerance and cold tolerance.—Hardy perennial. Tolerates high temperature to at least 30 degrees C. Tolerates low temperatures to degrees minus 25 C. No specific drought tolerance has not been observed.

What is claimed is:

1. A new and distinct variety of Iberis plant named ‘IBSZ0003’ substantially as illustrated and described herein.
